(19) United States
(12) Patent Application Publication
(10) Pub. No.: US 2022/0276183 A1
(43) Pub. Date: Sep. 1, 2022

(54) RADIATION IMAGING SYSTEM AND CONTROL APPARATUS

(71) Applicant: CANON KABUSHIKI KAISHA, Tokyo (JP)

(72) Inventor: Eriko Sato, Tokyo (JP)

(21) Appl. No.: 17/664,337

(22) Filed: May 20, 2022

Related U.S. Application Data
(63) Continuation of application No. PCT/JP2020/042963, filed on Nov. 18, 2020.

(30) Foreign Application Priority Data
Dec. 3, 2019 (JP) 2019-219078

(51) Int. Cl. G01N 23/04; H04N 5/378; H04N 5/369; G01N 23/06

(52) U.S. Cl. CPC G01N 23/04; H04N 5/378; H04N 5/3698; G01N 23/06; G01N 2223/04; G01N 2223/40

(57) ABSTRACT

A radiation imaging system comprising an imaging apparatus which includes a sensor and is capable of non-contact power reception, a power supplier capable of non-contact power supply to the imaging apparatus and a controller is provided. The imaging apparatus performs a first readout operation of reading out a signal accumulated in a period during which the sensor is irradiated with radiation and a second readout operation of reading out a signal accumulated in a period during which the sensor is not irradiated with radiation. In accordance with a temporal change in the power supplied from the power supplier to the imaging apparatus in a preceding operation of the first and second readout operations, the controller temporally changes the power supplied from the power supplier to the imaging apparatus in a succeeding operation of the first and second readout operations.

### CROSS-REFERENCE TO RELATED APPLICATIONS

This application is a Continuation of International Patent Application No. PCT/JP2020/042963, filed Nov. 18, 2020, which claims the benefit of Japanese Patent Application No. 2019-219078, filed Dec. 3, 2019, both of which are hereby incorporated by reference herein in their entirety.

### BACKGROUND OF THE INVENTION

### Field of the Invention

The present invention relates to a radiation imaging system and a control apparatus.

### Background Art

A radiation imaging system using a radiation imaging apparatus that obtains a radiation image by detecting the intensity distribution of radiation transmitted through an object and converting it into electric signals is broadly used. In the radiation imaging apparatus as described above, non-contact power supply is sometimes used in which the required power is received via a change in electromagnetic field from the outside. When the non-contact power supply is performed when reading out signals generated from the incident radiation by a sensor unit of the radiation imaging apparatus, a change in electromagnetic field by the non-contact power supply operation is superimposed on the signals, and this may degrade the image quality of the obtained radiation image. PTL 1 describes that the non-contact power supply is stopped during the period from the start of imaging to the end of A/D conversion of radiation image information based on the emitted radiation.

### CITATION LIST

### Patent Literature

PTL 1: Japanese Patent Laid-Open No. 2010-158515

If the power supply is stopped during the period of capturing a radiation image, it is necessary to install an internal power supply such as a battery in the radiation imaging apparatus. If the non-contact power supply is stopped, the internal power supply will not be charged, and the power may be run out during capturing a radiation image.

The present invention has as its object to provide a technique advantageous in performing non-contact power supply in a radiation imaging system.

### SUMMARY OF THE INVENTION

According to some embodiments, a radiation imaging system comprising a radiation imaging apparatus, which includes a sensor unit for obtaining a radiation image and is capable of non-contact power reception, a power supply apparatus capable of non-contact power supply to the radiation imaging apparatus, and a controller, wherein the radiation imaging apparatus is configured to perform a first readout operation of reading out, from the sensor unit, a signal accumulated in a period during which the sensor unit is irradiated with radiation, and a second readout operation of reading out, from the sensor unit, a signal accumulated in a period during which the sensor unit is not irradiated with radiation, and in accordance with a temporal change in the power supplied from the power supply apparatus to the radiation imaging apparatus in a period of a preceding operation, which is the precedingly performed readout operation of the first readout operation and the second readout operation, the controller is configured to temporally change the power supplied from the power supply apparatus to the radiation imaging apparatus in a period of a succeeding operation, which is the succeedingly performed readout operation of the first readout operation and the second readout operation, is provided.

According to some other embodiments, a control apparatus configured to control a radiation imaging apparatus, which comprises a sensor unit for obtaining a radiation image and is capable of non-contact power reception, and a power supply apparatus capable of non-contact power supply to the radiation imaging apparatus, wherein the radiation imaging apparatus is configured to perform a first readout operation of reading out, from the sensor unit, a signal accumulated in a period during which radiation irradiation is performed, and a second readout operation of reading out, from the sensor unit, a signal accumulated in a period during which no radiation irradiation is performed, and the control apparatus is configured to control the radiation imaging apparatus and the power supply apparatus such that the power supplied from the power supply apparatus to the radiation imaging apparatus in a period of a succeeding operation, which is the succeedingly performed readout operation of the first readout operation and the second readout operation, temporally changes in accordance with a temporal change in the power supplied from the power supply apparatus to the radiation imaging apparatus in a period of a preceding operation, which is the precedingly performed readout operation of the first readout operation and the second readout operation, is provided.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a view showing a configuration example of a radiation imaging system according to an embodiment;

FIG. 2 is a view showing an arrangement example of a radiation imaging apparatus of the radiation imaging system shown in FIG. 1;

FIG. 3 is a view showing an arrangement example of a power supply apparatus of the radiation imaging system shown in FIG. 1;

FIG. 4A is a graph for explaining the power supply of the radiation imaging system shown in FIG. 1;

FIG. 4B is a graph for explaining the power supply of the radiation imaging system shown in FIG. 1;

FIG. 5A is a flowchart illustrating a process during imaging performed in the radiation imaging system shown in FIG. 1;

FIG. 5B is a flowchart illustrating the process during imaging performed in the radiation imaging system shown in FIG. 1;

FIG. 6 is a timing chart illustrating an operation during imaging performed in the radiation imaging system shown in FIG. 1;

FIG. 7 is a flowchart illustrating a process during imaging performed in the radiation imaging system shown in FIG. 1;

FIG. 8 is a timing chart illustrating an operation during imaging performed in the radiation imaging system shown in FIG. 1; and

FIG. 9 is a timing chart illustrating an operation during imaging performed in the radiation imaging system shown in FIG. 1.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

Radiation in the present invention can include α-rays, β-rays, γ-rays, and the like which are beams generated by particles (including photons) emitted by radiation decay, as well as beams having the same or higher energy, for example, X-rays, particle beams, cosmic rays, and the like.

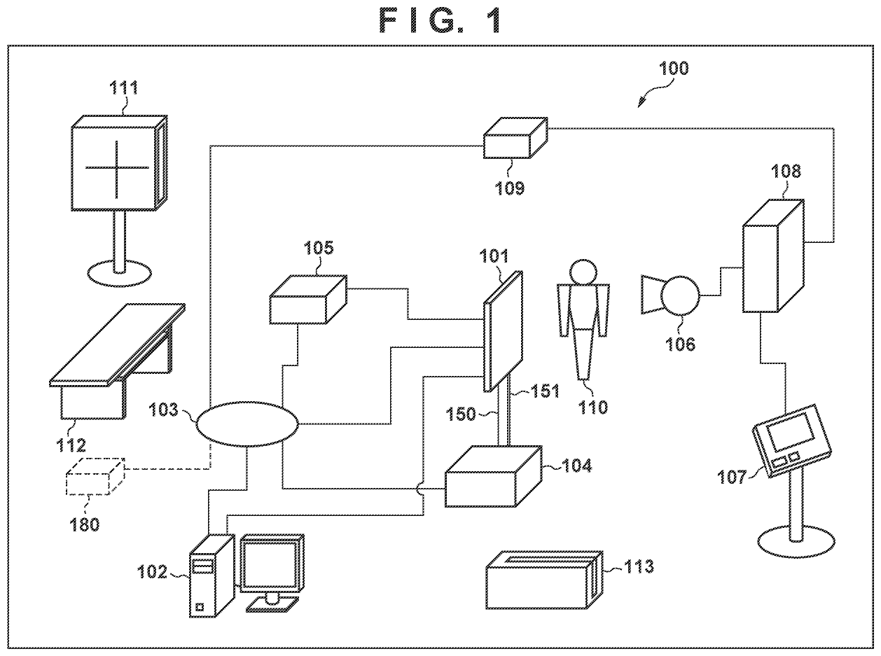

With reference to FIGS. 1 to 6, a radiation imaging system in some embodiments of this disclosure will be described. FIG. 1 shows a configuration example of a radiation imaging system 100 in a first embodiment of this disclosure. In this embodiment, a case will be described in which the radiation imaging system 100 operates in a synchronous imaging mode of performing imaging while synchronizing a radiation imaging apparatus 101 and a radiation generation apparatus 108. First, with reference to FIG. 1, respective components forming the radiation imaging system 100 and the relationships therebetween will be described below.

The radiation imaging apparatus 101 includes a sensor unit for obtaining a radiation image, and configured to be capable of non-contact power reception. The radiation imaging apparatus 101 includes a wired or wireless communication function or both a wired communication function and a wireless communication function, and can transmit/receive data to/from a console 102 via a communication path.

The console 102 is constructed by a personal computer (PC) or the like including a display function such as a monitor and a function of accepting an input from a user (for example, a technician). The console 102 can transfer an instruction from the user to the radiation imaging apparatus 101, and receive the image obtained by the radiation imaging apparatus 101 and display it to the user. The console 102 includes a wired or wireless communication function or both a wired communication function and a wireless communication function. In the configuration shown in FIG. 1, a stationary-type console is shown as the console 102, but the console 102 is not particularly limited in the actual operation of the radiation imaging system 100. For example, a portable-type notebook PC, a tablet equipment, or the like may be used as the console 102.

The radiation imaging apparatus 101 may transmit the obtained image data to the console 102 via either of a communication network 103, a power supply apparatus 104, and an access point (AP) 105, each forming a communication path, in accordance with the system configuration status. Further, the radiation imaging apparatus 101 may directly transmit the image data to the console 102. The communication network 103 is, for example, a LAN network, and can transmit/receive data when the radiation imaging apparatus 101 and the console 102 are connected to the communication network 103 using wired cables.

In this embodiment, the radiation imaging apparatus 101 includes a function capable of non-contact power reception. By bringing the power supply apparatus 104 capable of power supply to the radiation imaging apparatus 101 and the radiation imaging apparatus 101 close to each other, non-contact power supply from the power supply apparatus 104 to the radiation imaging apparatus 101 is enabled. Further, when each of the radiation imaging apparatus 101 and the power supply apparatus 104 includes a non-contact short-range communication function, each of a portion close to the non-contact power reception unit of the radiation imaging apparatus 101 and a portion close to the non-contact power supply unit of the power supply apparatus 104 may be provided with a component that performs communication. With this, when the radiation imaging apparatus 101 and the power supply apparatus 104 are brought close to each other, the radiation imaging apparatus 101 can receive power and perform communication via the power supply apparatus 104.

In the configuration shown in FIG. 1, between lines connecting the radiation imaging apparatus 101 and the power supply apparatus 104, a line 150 means a connection (wired and/or wireless) for communication, and a line 151 means a connection for power supply (non-contact power supply). In the configuration shown in FIG. 1, the form is shown in which the power supply apparatus 104 is connected to the console 102 via the communication network 103, but the present invention is not limited to this. The power supply apparatus 104 and the console 102 may be configured to be electrically connected directly to each other. Here, the electrical connection includes a connection for transmission/reception of data or the like between components connected to each other.

When the radiation imaging apparatus 101 includes a wireless communication function, the radiation imaging apparatus 101 may implement transmission/reception of data to/from the console 102 via the AP 105. In the configuration shown in FIG. 1, the form is shown in which the AP 105 is connected to the console 102 via the communication network 103, but the present invention is not limited to this. Similar to the power supply apparatus 104 described above, the AP 105 may be electrically connected directly to the console 102.

Further, when each of the radiation imaging apparatus 101, the console 102, the power supply apparatus 104, and the AP 105 includes a function of directly transmitting/receiving data with each other, they may directly transmit/receive data with each other in a wireless or wired manner.

The above is the description of the examples of paths between the radiation imaging apparatus 101 and the console 102 upon performing data transmission/reception.

Here, a cradle 113 as a charger of the radiation imaging apparatus 101 will be described. Although the internal arrangement of the radiation imaging apparatus 101 will be described later, the radiation imaging apparatus 101 includes an internal power supply such as a battery, and it is possible to charge the internal power supply mounted on the radiation imaging apparatus 101 by supplying power to the radiation imaging apparatus 101 from the outside. It is also possible to charge the internal battery by power reception from the power supply apparatus 104 described above. However, the radiation imaging system 100 may be prepared with the cradle 113 that can charge the internal battery by simply attaching the radiation imaging apparatus 101 thereto while capturing of a radiation image is not performed or the like.

A mechanism for supplying power from the cradle 113 to the radiation imaging apparatus 101 may be a power supply mechanism that requires an electric contact via a connector or the like, or may be a non-contact power supply mechanism. When the radiation imaging apparatus 101 is attached to the cradle 113, the cradle 113 detects the attachment of the radiation imaging apparatus 101 and enters a state capable of starting power supply. With this, the radiation imaging apparatus 101 can receive power and charge the internal power supply.

In the configuration shown in FIG. 1, the example is shown in which the cradle 113 does not communicate with other components of the radiation imaging system 100 and is arranged solely, but the present invention is not limited to this. The cradle 113 may include a communication function and be capable of communication such as data exchange with other components of the radiation imaging system 100 via the communication network 103 or the like. For example, while the radiation imaging apparatus 101 is attached to the cradle 113, the radiation imaging apparatus 101 may be capable of communication with the component such as the console 102 via the cradle 113. A plurality of the cradles 113 may be arranged in the radiation imaging system 100.

Next, the outline of imaging of an object 110 by radiation will be described. When imaging the object 110, the radiation imaging apparatus 101 is installed at a position where it receives the radiation emitted from a radiation tube 106 and transmitted through the object 110.

An example of the sequence of imaging will be described. After a user such as a technician activates the radiation imaging apparatus 101, the user operates the console 102 to set the radiation imaging apparatus 101 in a state capable of imaging. Then, the user operates a radiation generation apparatus console 107 to set the imaging conditions (the tube voltage of the radiation tube 106, the tube current, the irradiation time, and the like) for radiation irradiation. The imaging conditions for radiation irradiation may be set by operating the console 102. After the above processing is completed, the user confirms that the imaging preparation including the object 110 is ready and, for example, presses an exposure switch included in the radiation generation apparatus console 107 to expose radiation.

At the time of radiation exposure, the radiation generation apparatus 108 notifies, via a connection device 109 and the communication network 103, the radiation imaging apparatus 101 of a signal indicating that radiation irradiation is to be started. In the configuration shown in FIG. 1, the radiation imaging apparatus 101 and the radiation generation apparatus 108 are connected to each other via the connection device 109 and the communication network 103. However, the connection is not limited to this form and, as in the above description, the radiation imaging apparatus 101 and the radiation generation apparatus 108 may be connected directly to each other.

When the radiation imaging apparatus 101 receives the signal indicating that radiation irradiation is to be started, the radiation imaging apparatus 101 determines whether it is ready for radiation irradiation. If the radiation imaging apparatus 101 is ready for radiation irradiation and there is no problem for capturing a radiation image, it transmits an irradiation permission to the radiation generation apparatus 108. With this, radiation exposure is started.

When the radiation imaging apparatus 101 detects the end of radiation irradiation by various kinds of methods such as a notification from the radiation generation apparatus 108 or reference to a set time decided in advance, it starts to generate image data of a radiation image. The generated image data is transmitted to the console 102 via the communication path described above. The image data transmitted to the console 102 can be displayed as a radiation image on, for example, a display unit (for example, a display) included in the console 102.

In accordance with conditions such as the imaging part of the object 110 and the status of the object, the radiation imaging apparatus 101 may be incorporated in an imaging stand (gantry) 111 or a bed 112 to perform imaging.

The operation in the synchronous imaging mode of performing imaging while synchronizing the radiation imaging apparatus 101 and the radiation generation apparatus 108 has been described above.

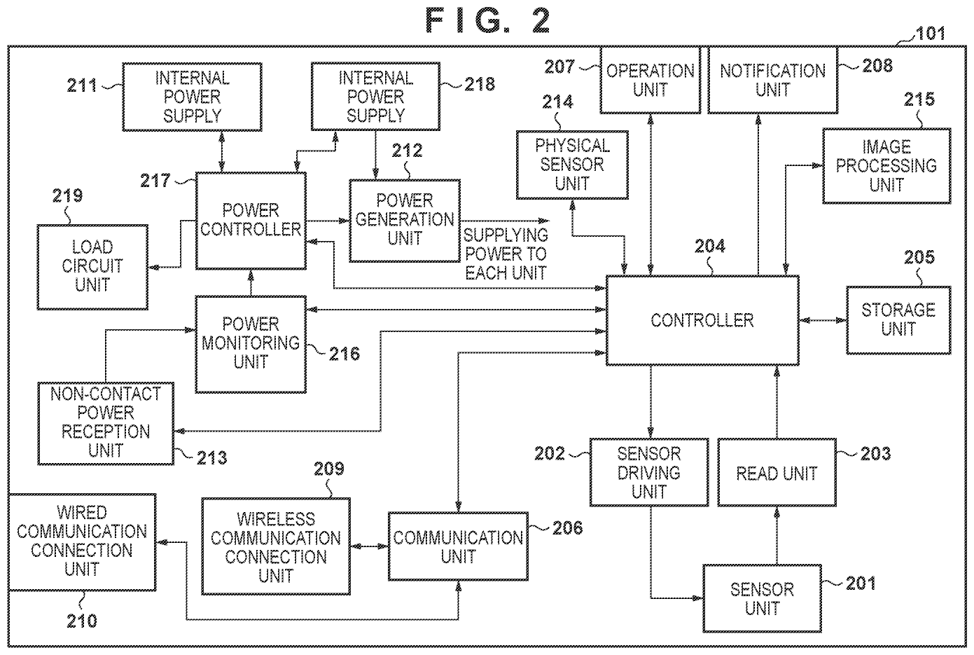

Next, the radiation imaging apparatus 101 will be described with reference to FIG. 2. FIG. 2 is a view showing an arrangement example of the radiation imaging apparatus 101. The radiation imaging apparatus 101 includes a sensor unit 201 that converts the incident radiation into electric signals to obtain a radiation image. The sensor unit 201 can be formed by including, for example, a scintillator for converting radiation into light, and an array of light detectors for detecting light converted by the scintillator. Each of the light detectors can be also called a pixel. Each of the scintillator and the light detector array has a two-dimensional plane shape, and they can be adjacent to each other with their surfaces facing each other. The scintillator is excited by radiation, and emits light (for example, visible light) detectable by the light detector. Electric charges corresponding to the intensity and duration of the light are accumulated in each light detector of the light detector array.

A sensor driving unit 202 drives the sensor unit 201 that detects radiation as electric charges. A reading unit 203 receives the electric charges output as a result of driving the sensor unit 201, and converts them into digital information. Upon extracting the accumulated electric charges, the sensor driving unit 202 selects, from the light detector array of the sensor unit 201, the light detectors to extract the signals. The reading unit 203 amplifies the signal charges extracted from the light detectors selected by the sensor driving unit 202, and then digitizes them.

The image data digitized by the reading unit 203 is transmitted to a controller 204, and the controller 204 transmits it to a storage unit 205. The image data stored in the storage unit 205 may be immediately transmitted to an external equipment via a communication unit 206. Alternatively, the image data may be transmitted to the external equipment via the communication unit 206 after undergoing some processing by the controller 204. The image data may be accumulated in the storage unit 205.

The controller 204 performs processing concerning the control of respective components of the radiation imaging apparatus 101. For example, the controller 204 outputs, to the sensor driving unit 202, an indication to drive the sensor unit 201 for imaging. Further, the controller 204 may drive to store the obtained image data in the storage unit 205, or may extract, from the storage unit 205, the image data stored in the storage unit 205 and transmit the image data to an external equipment via the communication unit 206.

Further, the controller 204 transmits image data to another equipment via the communication unit 206, and receives an indication from the console 102 or the like via the controller 206. The controller 204 also performs switching of activation/stop of the radiation imaging apparatus 101 or the like by a user operation of an operation unit 207. The controller 204 can also notify the user of an operation status or an error state via a notification unit 208. Further, although the details will be described later, the controller 204 controls a power controller 217 and the like arranged in the radiation imaging apparatus 101 to perform control so as to make constant the power received by the radiation imaging apparatus 101 in the period of capturing a radiation image.

In this embodiment, the above-described process contents are processed by one controller 204. However, the radiation imaging apparatus 101 may include a plurality of the controllers 204 each corresponding to the predetermined function, and the respective controllers 204 may share the process. The controller 204 can be implemented by various components such as a CPU, an MPU, an FPGA, and a CPLD, and there is no particular restriction on the specific implementation. The appropriate component may be selected in accordance with the function and performance required for the radiation imaging apparatus 101.

The storage unit 205 can be used to store the image data obtained by the radiation imaging apparatus 101, log information indicating the result of internal processing or the like, and the like. When the controller 204 is a CPU or the like which uses software, the storage unit 205 can also store the software for the controller 204 and the like. There is no restriction on the specific implement of the storage unit 205, and the storage unit 205 can be mounted in various combinations of volatile/nonvolatile storage devices such as various kinds of memories and an HDD. Further, although only one storage unit 205 is shown in the arrangement in FIG. 2, a plurality of the storage units 205 may be arranged in the radiation imaging apparatus 101.

The communication unit 206 performs processing for implementing communication between the radiation imaging apparatus 101 and other equipments forming the radiation imaging system 100. The communication unit 206 in this embodiment is connected to a wireless communication connection unit 209 for wireless communication, and can communicate with the console 102, the AP 105, and the like via the wireless communication connection unit 209. An example of the wireless communication connection unit 209 can be a wireless communication antenna. The communication unit 206 is also connected to a wired communication connection unit 210 and can communicate with the console 102 and the like via the wired communication connection unit 210. In the arrangement shown in FIG. 2, the wired communication connection unit 210 may be arranged in contact with the exterior of the radiation imaging apparatus 101 and connected via, for example a connector. Further, the wired communication connection unit 210 may have a function of short-distance non-contact communication. In this example, as an example of this, it will be described that the wired communication connection unit 210 is mounted with the function of short-distance non-contact communication including the components such as the power supply apparatus 104 to be described later. The communication unit 206 is not limited to the above-described form, and may be configured to perform wired communication alone or wireless communication alone. The communication standard and method are not particularly limited.

The radiation imaging apparatus 101 includes an internal power supply in the apparatus. More specifically, in this embodiment, the radiation imaging apparatus 101 includes two internal power supplies including an internal power supply 211 and an internal power supply 218. In this embodiment, the internal power supply 211 is a rechargeable battery, and is an internal power supply detachable from the radiation imaging apparatus 101. The internal power supply 218 is an undetachable rechargeable battery. During imaging or the like, if the power supply to the radiation imaging apparatus 101 is unintentionally cut off due to detachment of the internal power supply 211 or the like, the internal power supply 218 supplies power to each component requiring power and terminates the process in progress with the appropriate procedure. This can suppress the influence (damage) due to the sudden cutoff of the power to each component of the radiation imaging apparatus 101. The internal power supply 218 may not be used in normal imaging and the like but may function only in an emergency as described above. Further, for example, the internal power supply 218 may be a power supply having a smaller charge capacity than the internal power supply 211. In this embodiment, the radiation imaging apparatus 101 includes one internal power supply 211 and one internal power supply 218, which are two kinds of power supplies, but the present invention is not limited to this. The radiation imaging apparatus 101 may include only one internal power supply, or may include internal power supplies in various combinations of rechargeable, non-rechargeable, detachable, and undetachable power supplies. For example, the radiation imaging apparatus 101 may include two detachable internal power supplies 211 and one undetachable emergency internal power supply 218.

A power generation unit 212 generates, from the power supplied from the external and internal power supplies via the power controller 217, a voltage and a current required for each component of the radiation imaging apparatus 101, and distributes and supplies them. For example, when the radiation imaging apparatus 101 is close to the non-contact power supply function of the power supply apparatus 104 as the power from the outside, the radiation imaging apparatus 101 can receive the power supplied from the power supply apparatus 104 using a non-contact power reception unit 213. The power generation unit 212 uses the received power to supply power to each component of the radiation imaging apparatus 101.

The non-contact power reception unit 213 can perform non-contact power reception when it is close to the power supply apparatus 104 including the non-contact power supply function. Further, as has been described above, the non-contact power reception unit 213 may not only receive power but also transmit/receive information regarding non-contact power supply to/from the power supply apparatus 104 using the communication function.

In this embodiment, the non-contact power reception unit 213 can communicate with the power supply apparatus 104 using a power reception part (for example, a coil). Therefore, in the arrangement shown in FIG. 2, the non-contact power reception unit 213 is connected not only to the power controller 217 via a power monitoring unit 216 but also to the controller 204. As will be described later, the non-contact power reception unit 213 transmits, to the controller 204, the frequency information for supplying power from the power supply apparatus 104 to the radiation imaging apparatus 101. The communication between the non-contact power reception unit 213 and the controller 204 is not limited to this, and the communication between the non-contact power reception unit 213 and the controller 204 may be performed via the communication unit 206 or another component.

The operation unit 207 is used to accept an operation of the radiation imaging apparatus 101 from the user. The implementation method of the operation unit 207 is not particularly limited, and it is only required to accept an input from the user. For example, the operation unit 207 can be implemented by various kinds of switches or a touch panel operated by the user manually. Further, the operation unit 207 may include a receiving unit that accepts an input from a remote controller with which the user can operate the radiation imaging apparatus 101 away from the radiation imaging apparatus 101.

The notification unit 208 is used to notify the user or the like of the state of the radiation imaging apparatus 101 or the like. The implementation method of the notification unit 208 is not particularly limited, and it can be implemented by a lamp display using an LED or the like or a monitor display using an LCD or the like. Further, as one of the user notification methods, the notification unit 208 may include a sounding function such as a loudspeaker.

The radiation imaging apparatus 101 may include a physical sensor unit 214. The physical sensor unit 214 includes a sensor for detecting various kinds of physical events. Examples of the physical phenomena are the temperature, the acceleration, the terrestrial magnetism, and the electromagnetic field. Based on the detection information of the physical event, the controller 204 determines the status of the radiation imaging apparatus 101. In a case of a high temperature or a strong impact, the controller 204 may notify a warning via the notification unit 208. Further, the controller 204 may determine the installation orientation of the radiation imaging apparatus 101 based on the detection information of the physical event and transmit, to the user or the console 102, information (for example, a notification of the abnormal insertion direction into the stand 111) for improving the usability.

An image processing unit 215 performs image correction processing such as offset correction and gain correction on the image data converted into a digital value by the reading unit 203 or the image data stored in the storage unit 205. In the offset correction, the difference between the image data obtained with radiation irradiation and the image data obtained without radiation irradiation is calculated to remove the offset component generated due to a dark current regardless of the radiation irradiation. In the gain correction, in order to correct the gain variation among the respective light detectors (pixels), the obtained image data is corrected by dividing it by the image data captured while all the pixels are irradiated with uniform radiation. In general, higher image processing is often performed after transferring the image data to the console 102 or the like, but the present invention is not limited to this. The present invention does not limit the image processing contents performed in the radiation imaging apparatus 101.

The power monitoring unit 216 monitors a temporal change in the power supplied from the power supply apparatus 104 to the radiation imaging apparatus 101 via the non-contact power reception unit 213. Further, although the details will be described later, the power monitoring unit 216 is arranged to control the operation of the controller 204 performed to supply power from the power supply apparatus 104 to the radiation imaging apparatus 101.

The power controller 217 controls power supply in the radiation imaging apparatus 101 under the control of the controller 204. The power controller 217 takes a role of controlling and distributing, to each component, the power supplied from the internal power supplies 211 and 218 and the non-contact power reception unit 213. For example, when power is supplied from the power supply apparatus 104 via the non-contact power reception unit 213 and capturing of a radiation image is performed, the power controller 217 supplies the power to the sensor unit 201, the sensor driving unit 202, the reading unit 203, and the like via the power generation unit 212. For example, when power is supplied from the power supply apparatus 104 via the non-contact power reception unit 213 and no capturing is performed, the power controller 217 charges the internal power supplies 211 and 218.

In this embodiment, the radiation imaging apparatus 101 includes a load circuit unit 219. If the power supplied from the power supply apparatus 104 becomes excessive with respect to the power required in the radiation imaging apparatus 101, the power controller 217 supplies the power to the load circuit unit 219. The load circuit unit 219 may consume the power by converting the supplied power into heat. In this case, the load circuit unit 219 may include a resistor.

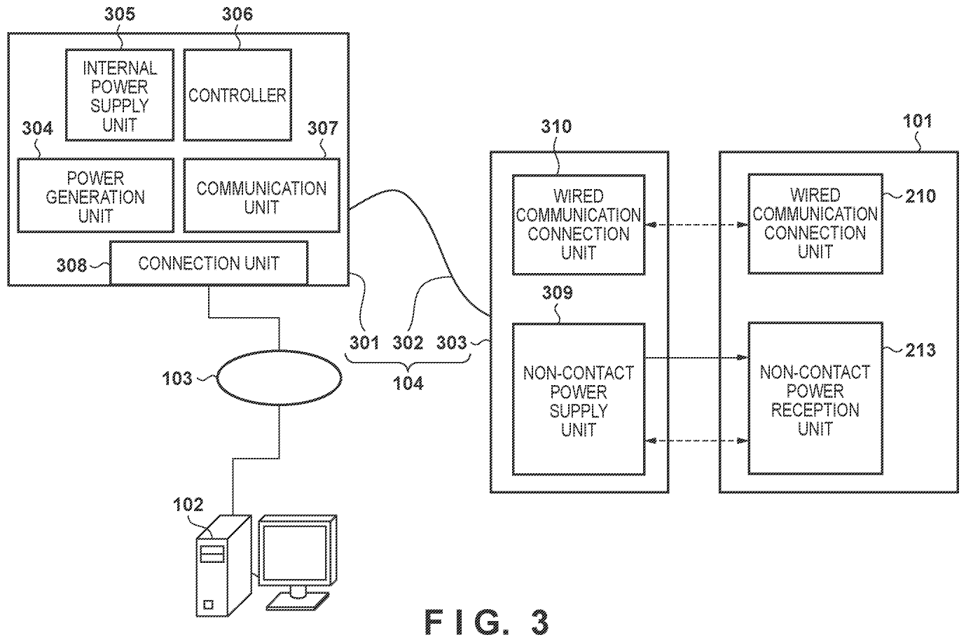

Next, the arrangement of the power supply apparatus 104 and an example of a connection between the radiation imaging apparatus 101 and the power supply apparatus 104 are shown in FIG. 3. FIG. 3 shows an example of the connection between the radiation imaging apparatus 101 and the power supply apparatus 104 and the information exchanged therebetween. In FIG. 3, in order to give attention to the portion regarding the non-contact power supply between the radiation imaging apparatus 101 and the power supply apparatus 104, the wired connection communication, and the short-distance non-contact communication, the communication path and connection form by another wireless connection will not be described. Further, FIG. 3 shows the power transfer and the information transmission.

The power supply apparatus 104 in this embodiment includes a power supply unit main body 301, a power supply unit cable 302, and a power supply unit proximity unit 303. When supplying power to the radiation imaging apparatus 101, the power supply unit proximity unit 303 is brought close to or into contact with the non-contact power reception unit 213 of the radiation imaging apparatus 101. The power supply unit main body 301 can be arranged in a location away from the radiation imaging apparatus 101 via the power supply unit cable 302. Here, “contact” is intended to bring the exterior of the radiation imaging apparatus 101 and the exterior of the power supply apparatus 104 into contact with each other.

The power supply unit main body 301 includes a power generation unit 304 that receives power from an AC power supply and converts the power into a DC voltage, and an internal power supply unit 305 that generates the power to be used by respective components in the power supply apparatus 104. The power supply unit main body 301 further includes a controller 306 that controls the respective components of the power supply apparatus 104, a communication unit 307 that performs communication between the power supply apparatus 104 and other components of the radiation imaging system 100, and a connection unit 308 used to perform communication with other than the radiation imaging apparatus 101.

The power supply unit proximity unit 303 includes a non-contact power supply unit 309 and a wired communication connection unit 310. The non-contact power supply unit 309 receives the power for power supply from the internal power supply unit 305, and the controller 306 controls the power supply therein. In this embodiment, similar to the non-contact power reception unit 213 of the radiation imaging apparatus 101, the non-contact power supply unit 309 performs communication regarding non-contact power supply using a power reception part (for example, a coil).

The wired communication connection unit 310 is a portion paired with the wired communication connection unit 210 of the radiation imaging apparatus 101 described above. As has been described above, since the wired communication connection unit 210 in this embodiment assumes short-distance non-contact communication, the corresponding wired communication connection unit 310 of the power supply apparatus 104 can have the arrangement and function similar to those of the wired communication connection unit 210. However, the portion regarding communication may be performed by a contact connection using a connector or the like. In order to perform communication, the wired communication connection unit 310 is connected to the communication unit 307 via the power supply unit cable 302.

In this embodiment, the case has been described in which the power supply unit main boy 301 and the power supply unit proximity unit 303 are placed at locations away from each other via the power supply unit cable 302. However, the present invention is not limited to this. The power supply unit proximity unit 303 may be formed to be incorporated in the power supply unit main body 301.

When the radiation imaging apparatus 101 receives power from the power supply apparatus 104, the power supply unit proximity unit 303 is brought close to the radiation imaging apparatus 101 in advance. For the sake of arrangement stability, the housing exterior of the radiation imaging apparatus 101 and that of the power supply apparatus 104 may be brought into contact with each other. When this state is set, the communication for mutual recognition between the non-contact power supply unit 309 and the non-contact power reception unit 213 is performed via the power supply coil and the power reception coil. When it is determined that power can be transferred between the radiation imaging apparatus 101 and the power supply apparatus 104, the power supply apparatus 104 supplies power to the radiation imaging apparatus 101 via the non-contact power supply unit 309. The radiation imaging apparatus 101 receives the power via the non-contact power reception unit 213 and uses the power in the radiation imaging apparatus 101.

When transferring the image data obtained by the radiation imaging apparatus 101 to an external equipment, the wired communication connection units 210 and 310 may be used to exchange the image data. For example, when transferring the image data obtained by the radiation imaging apparatus 101 to the console 102, the image data is transmitted to the console 102 from the radiation imaging apparatus 101 via the wired communication connection units 210 and 310, the communication unit 307, the connection unit 308, and the communication network 103.

Here, as has been repeatedly described, each connection is merely an example. Therefore, for example, the wired communication connection units 210 and 310 may use a method by contact via a connector, and the communication regarding non-contact power supply may be implemented using another path. Further, the image data is not limited to be transmitted from the radiation imaging apparatus 101 to the console 102 via the power supply apparatus 104, but may be directly transmitted to the console 102 from the radiation imaging apparatus 101.

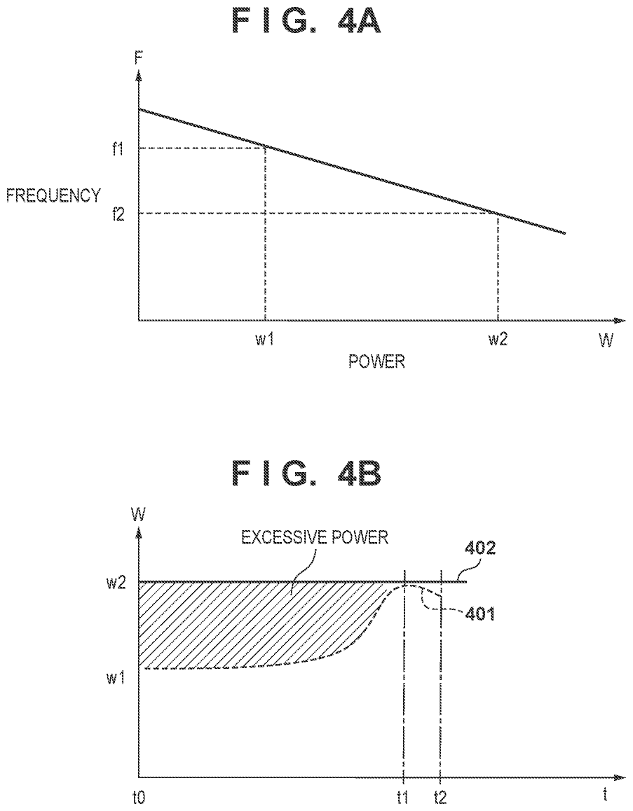

Next, the power supply in this embodiment will be described using FIGS. 4A and 4B. FIG. 4A is a graph showing the relationship between the power amount and the frequency upon supplying power from the power supply apparatus 104 to the radiation imaging apparatus 101 via the non-contact power reception unit 213. The power supply apparatus 104 supplies power to the radiation imaging apparatus 101 using a specific frequency band. In this embodiment, in the power supply apparatus 104, as the supplied power increases, the power supply frequency decreases. When no power is supplied, the frequency is highest.

FIG. 4B is a graph showing an example of the change in power consumption during the imaging operation of the radiation imaging apparatus 101. At time t0, the imaging operation is started by causing the sensor unit 201 to accumulate electric charges. Thereafter, at time t2, a readout operation of reading out the signals accumulated in the sensor unit 201 from the sensor unit 201 ends, and one imaging operation is completed. A dotted line 401 shown in FIG. 4B indicates the power supplied from the power controller 217 to the respective components via the power generation unit 212. In the period from time t0 to time t2, the power changes in accordance with the power usage status of the respective components. In the example shown in FIG. 4B, a minimum power w1 in one imaging operation is required at time t0, and a maximum power w2 in one imaging operation is required at time t1.

On the other hand, a solid line 402 indicates the power supplied from the power supply apparatus 104 to the power controller 217 of the radiation imaging apparatus 101 via the non-contact power reception unit 213 and the power monitoring unit 216 during the imaging operation. Although the details will be described later, in this embodiment, the storage unit 205 stores the maximum power of the power required when the radiation imaging apparatus 101 performs imaging. Therefore, the controller 204 obtains the information of the maximum power w2 stored in the storage unit 205. Then, during the imaging performed by the radiation imaging apparatus 101, the controller 204 controls such that the total power supplied to the respective units in the radiation imaging apparatus 101 via the power controller 217 becomes the maximum power w2. Hence, the power supply apparatus 104 supplies the power of the maximum power w2 to the radiation imaging apparatus 101 via the non-contact power reception unit 213 and the power supply unit proximity unit 303. Accordingly, the frequency for power supply from the power supply apparatus 104 to the radiation imaging apparatus 101 becomes constant during the period of the imaging operation performed by the radiation imaging apparatus 101.

Here, although the power supplied from the power controller 217 to the respective components via the power generation unit 212 changes over time (the power amount required by the respective components changes over time), the maximum power w2 is constantly supplied from the power supply apparatus 104. Hence, the excessive power occurs in the radiation imaging apparatus 101. If the power supplied from the power supply apparatus 104 is higher than the power used in the radiation imaging apparatus 101, for example, the power controller 217 may charge the internal power supplies 211 and 218 using the excessive power. For example, the internal power supplies 211 and 218 may be charged such that the power used in the radiation imaging apparatus 101 and the power supplied from the power supply apparatus 104 become equal to each other. Let Pt be the power supplied from the power supply apparatus 104 to the power controller 217, Pb1 be the power supplied from the power controller 217 to the internal power supply 211, Pb2 be the power supplied from the power controller 217 to the internal power supply 218, and Ps be the power supplied from the power controller 217 to the power generation unit 212. In this case,

Pt=Ps+Pb1+Pb2

holds.

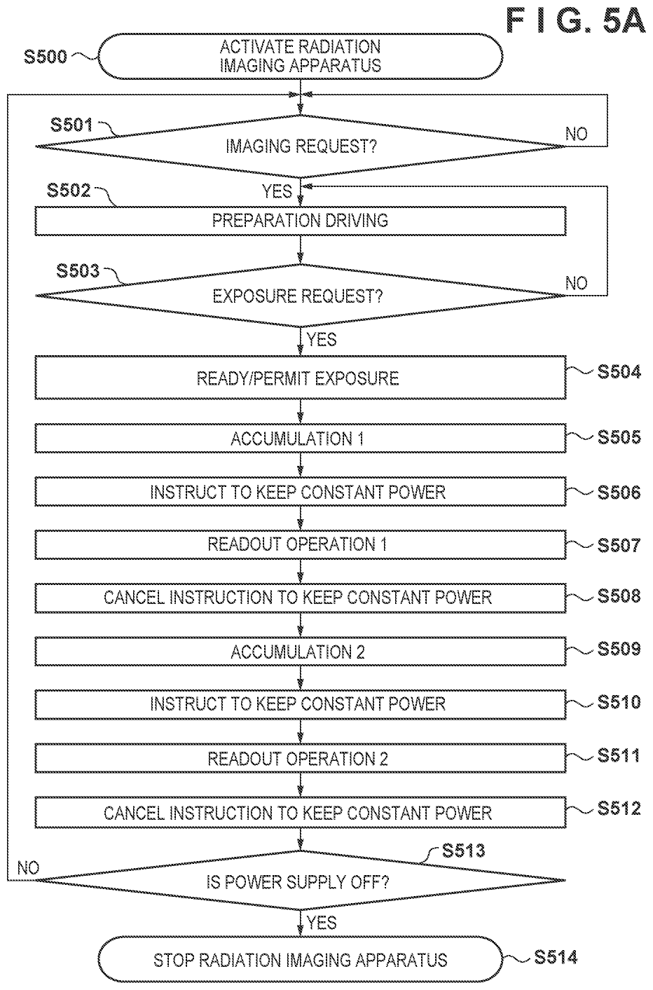

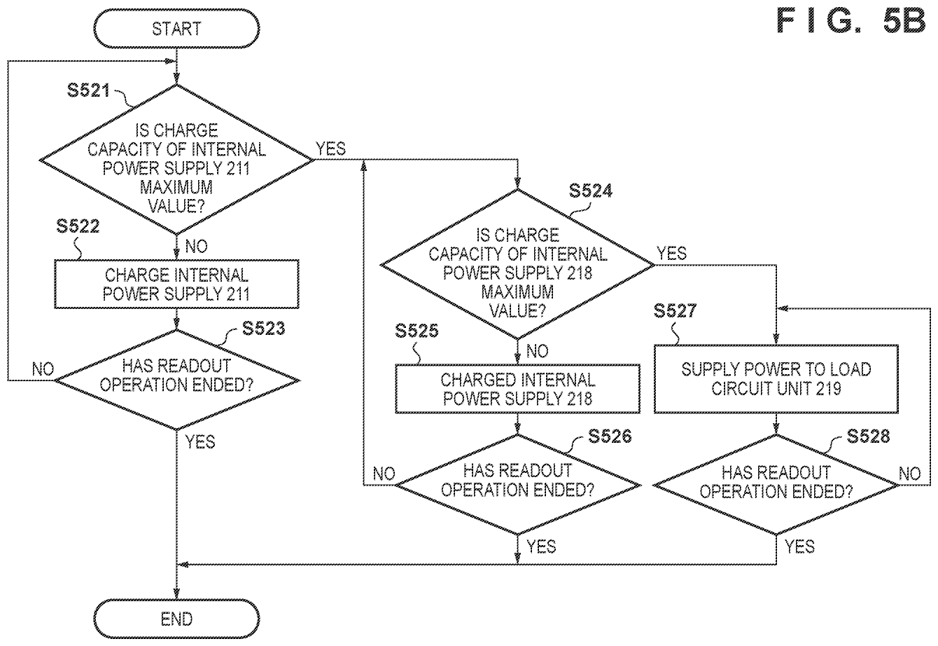

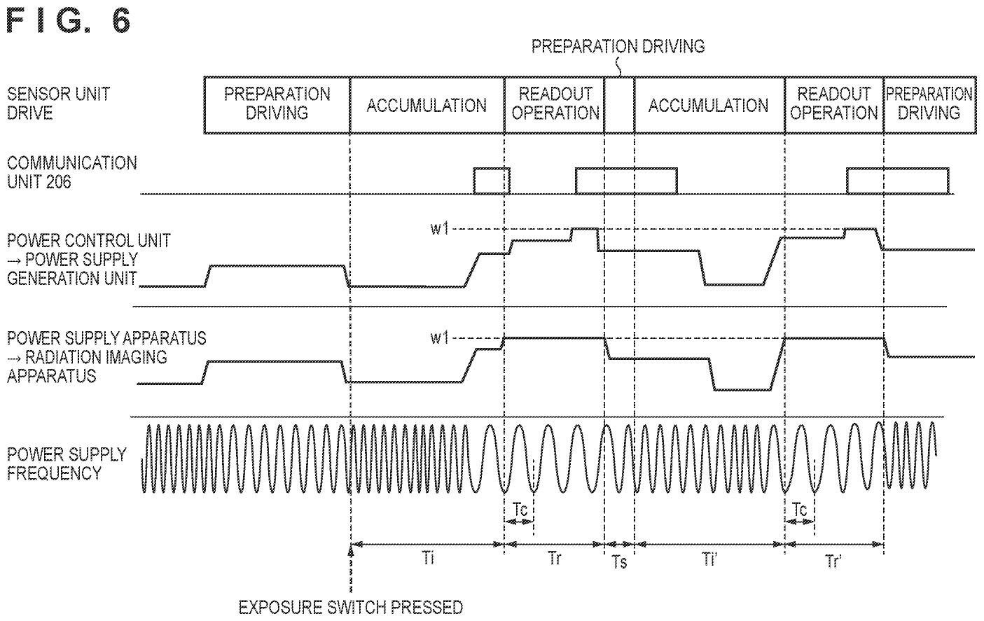

Next, the operation of the radiation imaging system 100 in a case of non-contact power supply will be described. In this embodiment, when performing non-contact power supply from the power supply apparatus 104 to the radiation imaging apparatus 101, the control to prevent fluctuation of the frequency for the non-contact power supply is performed during the readout operation of reading out the signals accumulated in the sensor unit 201 in the radiation imaging apparatus 101. FIGS. 5A and 5B are flowcharts illustrating a process during imaging performed in the radiation imaging system 100, and FIG. 6 is a timing chart illustrating an operation during imaging performed in the radiation imaging system 100. Here, it will be described that imaging is performed while synchronizing the radiation imaging apparatus 101 and the radiation generation apparatus 108.

When the radiation imaging apparatus 101 is activated by the user, power is supplied to the respective required components via the power generation unit 212 and the radiation imaging apparatus 101 starts (S500). At this time, the description will be given while assuming that the respective components of the radiation imaging system 100 required for imaging are also activated. For example, at the time of the activation of the radiation imaging apparatus 101 in S500, the power supply apparatus 104 is in a state capable of power supply to the radiation imaging apparatus 101. Detection of the user's activation intention by an operation of the operation unit 207 or by the physical sensor unit 214, attachment of the detachable internal power supply 211, connection of the power supply unit proximity unit 303 of the power supply apparatus 104 to the radiation imaging apparatus 101, or the like may be used as the activation trigger. At this time, it is unnecessary to activate all the components in the radiation imaging apparatus 101. For example, the component such as the sensor unit 201 used in imaging may not be activated until an imaging request is given. The radiation imaging apparatus 101 may be activated in accordance with a start of power supply from the outside to the radiation imaging apparatus 101 regardless of contact/non-contact. In this embodiment, the non-contact power supply from the power supply apparatus 104 to the radiation imaging apparatus 101 is continuously performed from the activation of the radiation imaging apparatus 101 in S500 of FIG. 5A to the stop of the radiation imaging apparatus 101 in S514.

When the radiation imaging apparatus 101 is activated, the process transitions to S501, and the controller 204 of the radiation imaging apparatus 101 determines whether an imaging request is given from the radiation generation apparatus 108. If there is no request, it continues to wait for an imaging request. If there is an imaging request, the process transitions to S502.

In S502, the radiation imaging apparatus 101 performs an imaging preparation operation. The controller 204 of the radiation imaging apparatus 101 supplies the power supplied from the power supply apparatus 104 to the sensor unit 201 via the power controller 217 and the power generation unit 212 to activate the sensor unit 201. Then, the controller 204 transmits, to the sensor driving unit 202, an instruction to start preparation driving. When the instruction is received, the sensor driving unit 202 performs the preparation driving of the sensor unit 201. The preparation driving may be an operation (reset operation) of continuously reading out the electric charges while scanning the detector array in the row direction to discharge the electric charges accumulated in the sensor unit 201 due to a dark current.

If the user presses the exposure switch of the radiation generation apparatus console 107 while the preparation driving is performed, communication is performed between the radiation generation apparatus 108 and the radiation imaging apparatus 101 via the connection device 109, the communication network 103, and the like. More specifically, the radiation generation apparatus 108 transmits an exposure request to the radiation imaging apparatus 101. If the radiation imaging apparatus 101 receives this request (YES in S503), the process transitions to S504. In S504, the radiation imaging apparatus 101 causes the sensor unit 201 to transition to a state of accumulating electric charges by radiation irradiation. When the radiation imaging apparatus 101 transmits a response indicating that the imaging can be performed or an exposure permission to the radiation generation apparatus 108, radiation irradiation is performed. While the user does not press the exposure switch (NO in S503), the above-described preparation driving is performed.

In S505, the radiation imaging apparatus 101 causes the sensor unit 201 to accumulate electric charges over a predetermined time during the radiation irradiation. When the radiation irradiation ends, the process transitions to S506. The controller 204 instructs, via the communication unit 206 and the communication unit 307, the power controller 217 to keep constant the power supplied to the respective units of the radiation imaging apparatus 101. In accordance with this, the power supply apparatus 104 supplies, to the radiation imaging apparatus 101 via the non-contact power reception unit 213 and the power supply unit proximity unit 303, the maximum power w2 which is required in the radiation imaging apparatus 101 during performing the readout operation of reading out the signals from the sensor unit 201 as illustrated in FIG. 6.

When the maximum power w2 is supplied from the power supply apparatus 104 to the radiation imaging apparatus 101, the process transitions to S507. In S507, the readout operation of reading out, from the sensor unit 201, the signals accumulated in the period during which the sensor unit 201 is irradiated with radiation is performed.

The power controller 217 controls the power supplied to the internal power supplies 211 and 218 and the power generation unit 212 such that the power used in the radiation imaging apparatus 101 becomes the maximum power w2. The controller 204 can calculate the excessive power based on the power supplied from the power controller 217 to the power generation unit 212 during the imaging operation with respect to the maximum power w2 stored in the storage unit 205 in advance. The power supplied from the power controller 217 to the power generation unit 212 may be measured by operating the sensor unit 201 and the like during S502, or may be stored in the storage unit 205 at another timing such as factory shipment or by a service tool.

As illustrated in FIG. 5B, if the power supplied from the power supply apparatus 104 is higher than the power used in the radiation imaging apparatus 101, that is, if the excessive power occurs, the power controller 217 charges the internal power supplies 211 and 218. First, the power controller 217 determines whether the charge capacity of the internal power supply 211 is the maximum value (S521). If the charge capacity of the internal power supply 211 is not the maximum value, the power controller 217 supplies the power to the internal power supply 211 to charge the internal power supply 211 (S522). Then, if the readout operation does not end (NO in S523), the process returns to S521. In S521, if the charge capacity of the internal power supply 211 is the maximum value, the power controller 217 determines whether the charge capacity of the internal power supply 218 is the maximum value (S524). If the charge capacity of the internal power supply 218 is not the maximum value, the power controller 217 supplies the power to the internal power supply 218 to charge the internal power supply 218 (S525). Then, if the readout operation does not end (NO in S526), the process returns to S524. In S524, if the charge capacity of the internal power supply 218 is the maximum value, that is, if both the charge capacity of the internal power supply 211 and the charge capacity of the internal power supply 218 are the maximum values, the power controller 217 supplies the power to the load circuit unit 219 (S527). Until the readout operation ends, the power controller 217 supplies the power to the load circuit unit 219 (NO in S528).

If the readout operation ends, the process transitions to S508 (YES in S523, S526, S528). In S508, the instruction given by the controller 204 in S506 to cause the power controller 217 to keep constant the power supplied to the respective units of the radiation imaging apparatus 101 is canceled.

Then, as illustrated in FIG. 6, the radiation imaging apparatus 101 performs an operation for correcting the dark current component by offset correction. More specifically, the radiation imaging apparatus 101 performs an imaging operation in which electric charges are accumulated in the sensor unit 201 without radiation irradiation and a readout operation of reading out, from the sensor unit 201, the signals accumulated in the period during which the sensor unit 201 is not irradiated with radiation is performed (S509 and S511). Also in the second imaging operation, as in S506, the controller 204 gives an instruction to keep constant the power supplied by the power controller 217 to the respective units of the radiation imaging apparatus 101.

At this time, in accordance with the temporal change in the power supplied from the power supply apparatus 104 to the power controller 217 of the radiation imaging apparatus 101 via the non-contact power reception unit 213 and the power monitoring unit 216 in the period of the preceding first readout operation (preceding operation, S507), the controller 204 temporally changes the power supplied from the power supply apparatus to the power controller 217 of the radiation imaging apparatus 101 via the non-contact power reception unit 213 and the power monitoring unit 216 in the period of the succeeding second readout operation (succeeding operation, S511). More specifically, in accordance with the temporal changes in frequency and phase of the power supplied from the power supply apparatus 104 to the power controller 217 of the radiation imaging apparatus 101 via the non-contact power reception unit 213 and the power monitoring unit 216 in the period of the first readout operation, the controller 204 temporally changes the frequency and phase of the power supplied from the power supply apparatus 104 to the power controller 217 of the radiation imaging apparatus 101 via the non-contact power reception unit 213 and the power monitoring unit 216 in the period of the second readout operation. For example, as illustrated in FIG. 6, the controller 204 controls the power controller 217 such that the temporal change in the power supplied to the radiation imaging apparatus 101 in the period of the second readout operation becomes equal to the temporal change in the power consumed in the respective units of the radiation imaging apparatus 101 via the power controller 217 in the period of the first readout operation. For example, as illustrated in FIG. 6, the controller 204 may adjust the operation timings of the power supply apparatus 104, the sensor driving unit 202, and the like so as to align the operation start phase in a period Tr, in which the first readout operation is performed and the operation start phase in a period Tr′ in which the second readout operation is performed. Here, it is described that the controller 204 adjusts the operation timings of the radiation imaging apparatus 101 and the power supply apparatus 104, but the present invention is not limited to this. For example, the controller 204 and the controller 306 may cooperate to adjust the operation timings, or the respective operation timings may be adjusted by a controller arranged outside (for example, the console 102) of the radiation imaging apparatus 101 and the power supply apparatus 104.

The adjustment of the operation timings may be performed by adjusting, for example, the period Tr (S507) of performing the first readout operation and a period Ti′ (S509) of the second accumulation of electric charges. Alternatively, the operation timings may be adjusted by adjusting a preparation driving period Ts in which a reset operation of resetting the sensor unit 201 before accumulating electric charges in the sensor unit 201 (S509) in the second imaging operation (S509 and S511) is performed.

In the first imaging operation, the controller 204 stores the frequency information using the power monitoring unit 216. Here, the start point of the waveform of a period Tc of the power received by the non-contact power reception unit 213 in the first imaging operation is obtained. Then, for example, the start point of the period Tc in the first readout operation (S507) and the start point of the period Tc in the second readout operation are adjusted by adjusting the period Ti′ of accumulating electric charges in the second imaging operation. At this time, if the power received by the non-contact power reception unit 213 becomes equal to the maximum power w2 by the time immediately before the end of the period Ti′, the frequency at the time of power supply has the period Tc, so that the phase can be aligned.

In this case, the time for accumulating electric charges in the sensor unit 201 in the first imaging operation and the time for accumulating electric charges in the sensor unit 201 in the second imaging operation may be different from each other. However, for the purpose of offset correction, the period Ti and the period Ti′ are required to be as close as possible. In non-contact power supply, the period Tc is often on the order of usec or less, and in capturing of a radiation image, each of the period Ti and the period Ti′ is generally on the order of msec or more. Therefore, even if the period Ti′ is adjusted, the accuracy of the offset correction is unlikely to be affected.

Further, the controller 204 may adjust the timing by using not the period Ti′ but the period Ts for the preparation driving. That is, the period Ts of the preparation driving, which is from the end of the first imaging operation to the start of accumulation of electric charges in the sensor unit 201 in the second imaging operation, may be changed in accordance with the period Ti for accumulating electric charges in the sensor unit 201 in the first imaging operation and the period Ti′ for accumulating electric changes in the sensor unit 201 in the second imaging operation. For example, the controller 204 may adjust the timing by making the time of the period Ts for the preparation driving longer than the minimum necessary time. Alternatively, the controller 204 may use both the period Ti′ and the period Ts to control to align the phase of the period Tc at the start of the first readout operation (S507) and the phase of the period Tc at the start of the second readout operation (S509).

It has been described that in the above-described operation, the controller 204 arranged in the radiation imaging apparatus 101 adjusts the length of the period Ts of the preparation driving and the period Ti′ which is the accumulation time in the second imaging operation, but the present invention is not limited to this. The radiation generation apparatus console 107 or the console 102 may determine the lengths of the periods Ts and Ti′ in accordance with the imaging condition concerning radiation irradiation input to the radiation generation apparatus console 107 by the user. The periods Ts and Ti′ determined by the radiation generation apparatus console 107 or the console 102 are transmitted to the radiation imaging apparatus 101 and the power supply apparatus 104. For example, the radiation generation apparatus console 107 or the console 102 may notify the settings concerning the above-described timing when the radiation imaging apparatus 101 is activated. Further, as shown in FIG. 1, a control apparatus 180 for controlling the radiation imaging apparatus 101 capable of non-contact power reception and the power supply apparatus 104 capable of non-contact power supply to the radiation imaging apparatus 101 may be arranged in the radiation imaging system 100 independently of the above-described respective components.

By performing the control as described above, noise components of the same frequency are superimposed with the same phase on the image data obtained in the readout operation of the first imaging operation and the image data obtained in the readout operation of the second imaging operation, respectively. Accordingly, by performing offset correction, when reading out the signals generated in the sensor unit 201, the noise superimposed on the signal due to the change in the electromagnetic field caused by the non-contact power supply from the power supply apparatus 104 to the radiation imaging apparatus 101 is canceled. That is, the controller 204 temporally changes the power supplied from the power supply apparatus 104 to the radiation imaging apparatus 101 in the period of the second readout operation such that the periodic change in noise caused by the non-contact power supply from the power supply apparatus 104 to the radiation imaging apparatus 101 and superimposed on the signal read out in the first readout operation is reduced by obtaining the difference from the signal read out in the second readout operation. This suppresses a degradation in image quality of the obtained radiation image. In addition, since the non-contact power supply is continued even during the imaging, for example, it is suppressed that the imaging cannot be continued due to the insufficient charging of the internal power supplies 211 and 218 of the radiation imaging apparatus 101 during the imaging. Further, since the non-contact power supply is continuously performed during the imaging, the capacity of the internal power supply of the radiation imaging apparatus 101 can be reduced, and the imaging can be performed even without the internal power supply. Therefore, a reduction of the weight of the radiation imaging apparatus 101 and the like are possible.

In this embodiment, it has been described that the signals accumulated in the period during which radiation irradiation is performed are obtained in the first imaging operation (S505 and S507), and the signals accumulated in the period during which no radiation irradiation is performed are obtained in the second imaging operation (S509 and S511). However, the present invention is not limited to this. No radiation irradiation may be performed in S505 and radiation irradiation may be performed in S509.

When the readout operation is completed in S511, the process transitions to S512. In S512, as in S508, the instruction given in S510 is canceled. Then, the radiation imaging apparatus 101 transmits, to the console 102 via the communication network 103 or the like, the image data having undergone correction processing such as predetermined offset correction, and the imaging operation is completed. Alternatively, the image data may be not transmitted to the console 102 but stored in the storage unit 205.

Thereafter, if an instruction to turn off the radiation imaging apparatus 101 is given by the user by operating the operation unit 207 (YES in S513), the radiation imaging apparatus 101 is turned off, the process transitions to S514, and the radiation imaging apparatus 101 is set in a stop state. If no instruction to turn off the radiation imaging apparatus 101 is given (No in S513), the process returns to S501, and the radiation imaging apparatus 101 waits until the next imaging request is received. Further, in S501, if the standby time becomes long, the radiation imaging apparatus 101 may enter a sleep state in which the power consumption is suppressed by turning off the display of the notification unit 208 or the like, or the process may transition to S514 to turn off the radiation imaging apparatus 101.

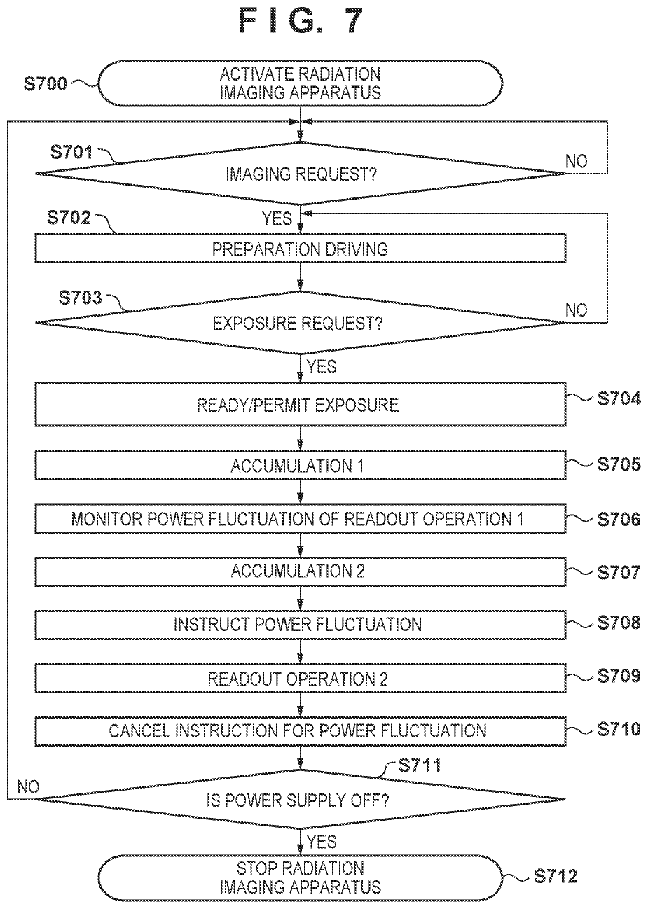

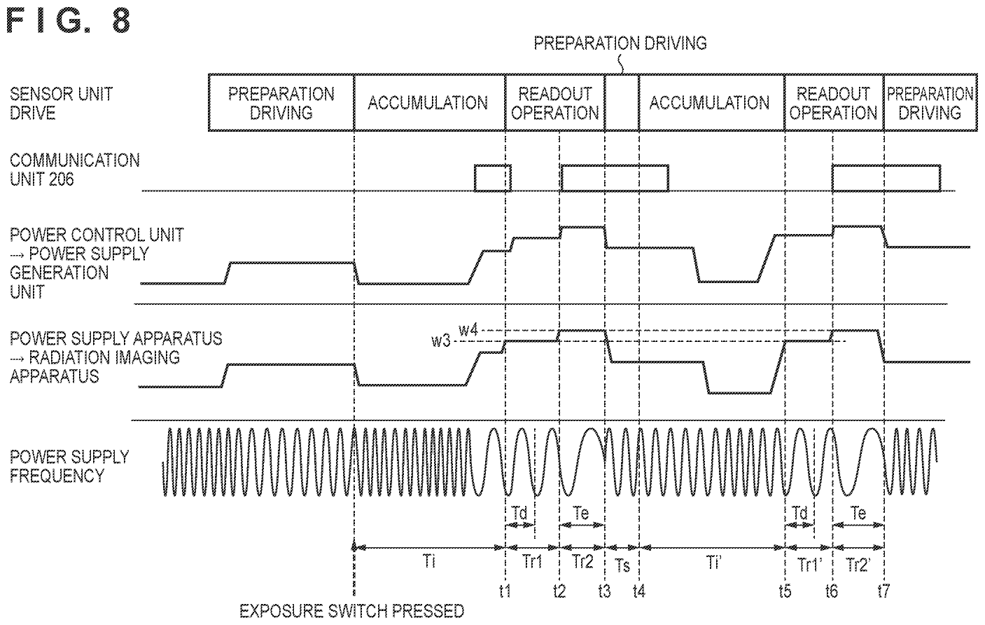

With reference to FIGS. 7 and 8, an operation of a radiation imaging system 100 according to a second embodiment of this disclosure will be described. FIG. 7 is a flowchart illustrating a process during imaging performed in the radiation imaging system 100, and FIG. 8 is a timing chart illustrating an operation during imaging performed in the radiation imaging system 100. The configuration of the radiation imaging system 100 may be similar to that in the first embodiment described above, so that the description thereof will be omitted.

In this embodiment, during the non-contact power supply from a power supply apparatus 104 to a radiation imaging apparatus 101, even if the frequency for the non-contact power supply temporally changes during the period of the first readout operation of image data of the radiation imaging apparatus 101, a controller 204 controls such that the temporal change in the frequency for the non-contact power supply during the period of the second readout operation matches that in the first readout operation. Also in this embodiment, the non-contact power supply is continuously performed from the activation of the radiation imaging apparatus in S700 of FIG. 7 to the stop of the radiation imaging apparatus in S712.

In the process illustrated in FIG. 7, S700 to S705 are similar to S500 to S505 described above, so that the description thereof will be omitted here. When the operation of accumulating electric charges is completed in S705, the process transitions to S706, and the first readout operation is performed. In S706, in accordance with the power supplied from a power controller 217 to respective units of the radiation imaging apparatus 101, a power monitoring unit 216 transmits, to the controller 204, information of the temporal change in the power received by a non-contact power reception unit 213 from the power supply apparatus 104. The information to be transmitted includes the power amount and the time at which the power amount has changed. Further, the controller 204 stores the operations (for example, communication) of the respective units that have caused the change in the power received by the non-contact power reception unit 213.

When reading out of the signals ends, then, in order to correct the dark current component by offset correction, the radiation imaging apparatus 101 causes a sensor unit 201 to accumulate electric charges over a predetermined time (S707). Then, based on the information of the temporal change in the power stored in S706, the controller 204 gives a power fluctuation instruction immediately before the second imaging operation (S708). For example, the controller 204 transmits the information of the temporal change in the power to the power controller 217. In accordance with the information of the temporal change in the power obtained in the first readout operation by the power monitoring unit 216, the power controller 217 supplies the power to the respective units of the radiation imaging apparatus 101 in the second readout operation. In accordance with the power consumed in the radiation imaging apparatus 101, the power supply apparatus 104 supplies power to the radiation imaging apparatus 101 via the non-contact power reception unit 213.

In FIG. 8, during the period of the first readout operation (time t1 to time t3), the power supplied from the power supply apparatus 104 to the radiation imaging apparatus 101 fluctuates at time t2, and a period Td of the power supply frequency in a period Tr1 from time t1 to time t2 changes to a period Te in a period Tr2 from time t2 to time t3. In this case, during the period (time t5 to time t7) of the second readout operation, the power supply apparatus 104 is controlled such that the supplied power changes at time t6. Letting a period Tr1′ be the period from time t5 to time t6 and a period Tr2′ be the period from time t6 to time t7, Tr1=Tr1′ and Tr2=Tr2′ can hold. In addition, since the power amount supplied from the power supply apparatus 104 to the radiation imaging apparatus 101 in the period Tr1 and that in the period Tr1′ become equal to each other, the power supply frequency has the period Td. Similarly, since the power amount supplied from the power supply apparatus 104 to the radiation imaging apparatus 101 in the period Tr2 and that in the period Tr2′ become equal to each other, the power supply frequency has the period Te.

For example, when the power supplied from the power supply apparatus 104 to the radiation imaging apparatus 101 in the period Tr1 is a power w3, the power controller 217 controls the power supplied to the respective units of the radiation imaging apparatus 101 such that the power w3 is supplied from the power supply apparatus 104 to the radiation imaging apparatus 101 also in the period Tr1′. In the operation example illustrated in FIG. 8, the communication by a communication unit 206 is performed at the start of the first readout operation but no communication by the communication unit 206 is performed at the start of the second readout operation. Therefore, if the power w3 is supplied at the start of the second readout operation, an excessive power occurs. In this case, as in the first embodiment described above, the power controller 217 charges internal power supplies 211 and 218 or supplies the power to a load circuit unit 219 to make the power used in the radiation imaging apparatus 101 equal to the power supplied from the power supply apparatus 104. Alternatively, for example, the controller 204 may operate the communication unit 206 at the start of the second readout operation to increase the power used in the radiation imaging apparatus 101.

To the contrary, it is also conceivable that the power consumed in the period from time t5 to time t7 in the second readout operation becomes higher than the power w3 or a power w4 consumed in the period from time t1 to time t3 in the first readout operation. If the power supplied from the power supply apparatus 104 is lower than the power used in the radiation imaging apparatus 101, the power controller 217 may cause the internal power supplies 211 and 218 to supply power in parallel with the power supply apparatus 104. For example, the power controller 217 causes the internal power supplies 211 and 218 to supply power so as to make the power supplied from the power supply apparatus 104 and the internal power supplies 211 and 218 equal to the power used in the radiation imaging apparatus 101. If the power supplied from the power supply apparatus 104 is changed, the frequency for power supply from the power supply apparatus 104 changes from that in the first readout operation. Therefore, the power is supplied from the internal power supplies 211 and 218, and this can improve the accuracy of the offset correction without the change in the frequency.

Further, as in the first embodiment described above, the controller 204 adjusts the timing such that the phase at the start of the first readout operation with respect to the period Td of the power reception frequency in the first readout operation and the phase at the start of the second readout operation with respect to the period Td of the power reception frequency in the second readout operation are aligned with each other. The phase of the period Te in the period Tr2 and that in the period Tr2′ can be aligned with each other by aligning the times of changing the power supplied from the power supply apparatus 104 to the radiation imaging apparatus 101.

When the second readout operation in S709 ends, the instruction given in S708 is canceled in S710. Thereafter, as in the first embodiment described above, transmission of the image data and the like are performed, and the radiation imaging apparatus 101 is turned off or stands by in a stop state in accordance with a user instruction.

By performing the control as described above, also in this embodiment, noise components of the same frequency are superimposed with the same phase on the image data obtained in the first imaging operation and the image data obtained in the second imaging operation, respectively. Accordingly, by performing offset correction, when reading out the signals generated in the sensor unit 201, the noise superimposed on the signal due to the change in the electromagnetic field caused by the non-contact power supply operation is canceled. This suppresses a degradation in image quality of the obtained radiation image.

As has been described above, in each embodiment described above, the controller 204 controls the respective units of the radiation imaging apparatus 101 such as the power controller 217 such that the power supply frequencies for power supply from the power supply apparatus 104 to the radiation imaging apparatus 101 are aligned with each other in two or more periods in each of which the signal read out from the sensor unit 201 by the radiation imaging apparatus 101 is affected by the fluctuation of the power reception frequency. The period during which the signals read out from the sensor unit 201 by the radiation imaging apparatus 101 are affected by the fluctuation of the power reception frequency can be the period of reading out the signals to generate a radiation image as described above. In the period of reading out the signals to generate a radiation image, the radiation imaging apparatus 101 causes the pixels of the sensor unit 201 to accumulate electric charges over a predetermined time, and reads out the signals corresponding to the accumulated electric charges. Here, the period of causing the pixels to accumulate electric charges and reading out the signals corresponding to the accumulated electric charges may be a period of accumulating electric charges generated by radiation irradiation to generate a radiation image and reading out a signal corresponding to accumulated the electrical charges. Alternatively, the period of causing the pixels to accumulate electric charges and reading out the signals corresponding to the accumulated electric charges may be a period for obtaining image data to perform offset correction upon generating a radiation image. With these operations, the radiation imaging system 100 according to this embodiment can always perform non-contact power supply from the power supply apparatus 104 to the radiation imaging apparatus 101 while suppressing the influence on the image quality of a radiation image.

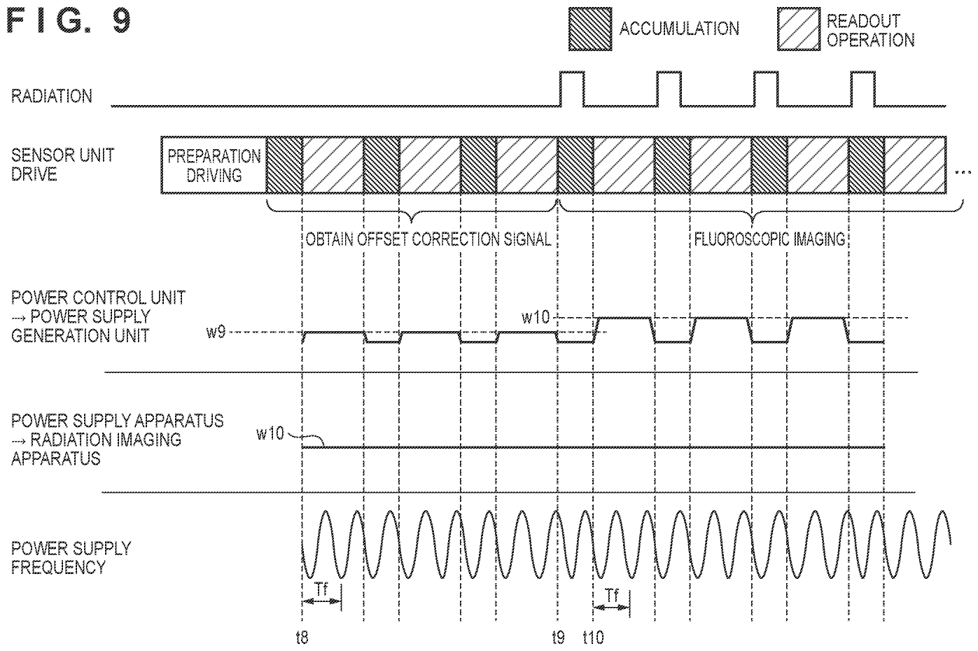

Further, capturing a radiation image performed in the radiation imaging system 100 is not limited to capturing a still image as described above. FIG. 9 is a timing chart of a case in which the radiation imaging apparatus 101 performs fluoroscopic imaging (moving image capturing) while non-contact power supply from the power supply apparatus 104 to the radiation imaging apparatus 101 is performed.

When performing fluoroscopic imaging, the radiation imaging apparatus 101 reads out, in real time, the signals generated by continuous radiation exposure. In FIG. 9, after preparation driving is performed, an operation of continuously performing imaging operations, in each of which electric charges are accumulated in the sensor unit 201 without irradiation radiation and then a readout operation of reading out the signals accumulated in the sensor unit 201 is performed, is performed in the period from time t8 to time t9. Then, after time t10, an operation of continuously performing imaging operations, in each of which electric charges are accumulated in the sensor unit 201 during irradiation radiation and then a readout operation of reading out the signals accumulated in the sensor unit 201 is performed, is performed. The signals read out in the period from time t8 to time t9 are used to correct (offset correct) the dark current components of the signals obtained after time t10. As illustrated in FIG. 9, when a plurality of offset correction signals are obtained, the signals may be averaged. Alternatively, the average value or the noise of the image or the like may be measured, and only appropriate signals may be extracted to use for correction. In the example shown in FIG. 9, the signals accumulated without radiation irradiation are obtained three times, but the number of times is not limited to three. The number of times can change in accordance with each mode of the subsequent fluoroscopic imaging. Here, the imaging mode may be the frame rate of imaging depending on the part to be captured or the method. For example, the number of times of obtaining the signals used for correction may be one or two, or may be four or more. It is only required that the correction signals are obtained at least once.

Assume that in the fluoroscopic imaging, as illustrated in FIG. 9, the power supplied from the power controller 217 to the power generation unit 212 is a power w9 when obtaining correction signals and is a power w10 during the fluoroscopic imaging. In this case, if the power supply apparatus 104 supplies power to the radiation imaging apparatus 101 in accordance with the power required by the radiation imaging apparatus 101, the power supply frequency temporally changes in accordance with the power required by the radiation imaging apparatus 101. In this case, the frequency of noise superimposed on the image data changes between the imaging for obtaining correction signals and the fluoroscopic imaging, so the noise cannot be canceled by the correction using a dark image during the fluoroscopic imaging.

In order to superimpose the noise components of the same frequency on the respective image data, as in each embodiment described above, a constant power is supplied from the power supply apparatus 104 to the radiation imaging apparatus 101. Therefore, a storage unit 205 stores, in advance, information such as the power necessary for the readout operation corresponding to each imaging mode set in the fluoroscopic imaging and the frequency at the power supply corresponding to the power. In accordance with the information stored in the storage unit 205, the controller 204 controls the power supplied to the respective units of the radiation imaging apparatus 101 by the power controller 217. The information may be stored at the time of factory shipment or by a service tool.

In accordance with an instruction of the fluoroscopic imaging mode from the console, the controller 204 controls the power controller 217 such that the power w10 required for the readout operation in the fluoroscopic imaging is supplied from the start of the readout operation of the dark image. During the fluoroscopic imaging, the accumulation time is several ms or more. Hence, if the power supply apparatus 104 supplies a constant power not only in the readout operation but also in the period of accumulating electric charges, the power control is facilitated. However, in a case of the fluoroscopic imaging with a long accumulation time and a low frame rate, the power supply amount in the period of accumulating electric charges may be different from that in the readout operation.

As illustrated in FIG. 9, also in the fluoroscopic imaging, there can be the time at which the power w10 supplied from the power supply apparatus 104 is higher than the power consumed in the radiation imaging apparatus 101 so that an excessive power occurs. Also in the fluoroscopic imaging, as in each embodiment described above, if the power supplied from the power supply apparatus 104 is higher than the power used in the radiation imaging apparatus 101, the power controller 217 may charge the internal power supplies 211 and 218. Further, if the power supplied from the power supply apparatus 104 is higher than the power used in the radiation imaging apparatus 101, the power controller 217 may cause the load circuit unit 219 to consume the excessive power.

Further, as illustrated in FIG. 9, the controller 204 adjusts the timing such that the phase at the start of the readout operation of reading out the signal of the dark current component and the phase at the start of each readout operation in the fluoroscopic imaging are aligned with each other with respect to a period Tf of the power supply frequency. For example, based on the frequency corresponding to the power to be supplied, which is stored in the storage unit 205, the controller 204 adjusts the accumulation time of accumulating electric charges such that the phase in the period Tf is aligned. Since the period Tf of the frequency is sufficiently shorter than the accumulation time, the influence on the necessary frame rate is small.

By performing the control as described above, also in the fluoroscopic imaging, noise components of the same frequency are superimposed with the same phase on the image data obtained in the readout operation of obtaining the dark current component for offset correction and the image data obtained in the readout operation for the fluoroscopic imaging, respectively. Accordingly, by performing offset correction, when reading out the signal generated in the sensor unit, the noise superimposed on the signal due to the change in the electromagnetic field caused by the non-contact power supply operation is canceled. This suppresses a degradation in image quality of the obtained radiation image. In addition, during continuous imaging such as the fluoroscopic imaging in which the imaging time can be longer and the more power can be consumed than in the still image capturing described above, the power can be constantly supplied from the power supply apparatus 104 to the radiation imaging apparatus 101 in the non-contact manner. Accordingly, the possibility of running out the power is suppressed, so that the user-friendly radiation imaging system 100 can be provided.

The above-described means provides a technique advantageous in performing non-contact power supply in a radiation imaging system.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging system comprising a radiation imaging apparatus, which includes a sensor unit for obtaining a radiation image and is capable of non-contact power reception, a power supply apparatus capable of non-contact power supply to the radiation imaging apparatus, and a controller, wherein
the radiation imaging apparatus is configured to perform a first readout operation of reading out, from the sensor unit, a signal accumulated in a period during which the sensor unit is irradiated with radiation, and a second readout operation of reading out, from the sensor unit, a signal accumulated in a period during which the sensor unit is not irradiated with radiation, and
in accordance with a temporal change in the power supplied from the power supply apparatus to the radiation imaging apparatus in a period of a preceding operation, which is the precedingly performed readout operation of the first readout operation and the second readout operation, the controller is configured to temporally change the power supplied from the power supply apparatus to the radiation imaging apparatus in a period of a succeeding operation, which is the succeedingly performed readout operation of the first readout operation and the second readout operation.

2. The radiation imaging system according to claim 1, wherein
in accordance with temporal changes in frequency and phase of the power supplied from the power supply apparatus to the radiation imaging apparatus in the period of the preceding operation, the controller is configured to temporally change a frequency and a phase of the power supplied from the power supply apparatus to the radiation imaging apparatus in the period of the succeeding operation.

3. The radiation imaging system according to claim 1, wherein
the controller is configured to temporally change the power supplied from the power supply apparatus to the radiation imaging apparatus in the period of the succeeding operation such that a periodic change in noise caused by the power supply from the power supply apparatus to the radiation image apparatus and superimposed on the signal read out in the preceding operation is reduced by obtaining a difference from the signal read out in the succeeding operation.

4. The radiation imaging system according to claim 1, wherein
the controller is configured to cause the power supply apparatus to supply the power to the radiation imaging apparatus such that the temporal change in the power supplied from the power supply apparatus to the radiation imaging apparatus in the period of the succeeding operation becomes equal to the temporal change in the power supplied from the power supply apparatus to the radiation imaging apparatus in the period of the preceding operation.

5. The radiation imaging system according to claim 1, further including
a storage unit configured to store a maximum power of power required in the period of the first readout operation and the period of the second readout operation,
wherein the controller is configured to cause the power supply apparatus to supply, to the radiation imaging apparatus, the maximum power stored in the storage unit in the period of the first readout operation and the period of the second readout operation.

6. The radiation imaging system according to claim 1, further comprising
a power monitoring unit configured to obtain information of the temporal change in the power supplied from the power supply apparatus to the radiation imaging apparatus in the preceding operation,
wherein in the succeeding operation, the controller is configured to cause power supply in accordance with the information of the temporal change in the power obtained by the power monitoring unit in the preceding operation.

7. The radiation imaging system according to claim 6, wherein
the radiation imaging apparatus includes an internal power supply and a power controller configured to control power supply in the radiation imaging apparatus, and
in the period of the first readout operation and the period of the second readout operation, if the power supplied from the power supply apparatus is lower than a power used in the radiation imaging apparatus, the power controller is configured to cause the internal power supply to supply power in parallel with the power supply apparatus.

8. The radiation imaging system according to claim 7, wherein
in the period of the first readout operation and the period of the second readout operation, if the power supplied from the power supply apparatus is lower than the power used in the radiation imaging apparatus, the power controller is configured to cause the internal power supply to supply power such that the power supplied from the power supply apparatus and the internal power supply becomes equal to the power used in the radiation imaging apparatus.

9. The radiation imaging system according to claim 7, wherein
in the period of the first readout operation and the period of the second readout operation, if the power supplied from the power supply apparatus is higher than the power used in the radiation imaging apparatus, the power controller is configured to charge the internal power supply.

10. The radiation imaging system according to claim 1, wherein
the radiation imaging apparatus comprises an internal power supply and a power controller configured to control power supply in the radiation imaging apparatus, and
in the period of the first readout operation and the period of the second readout operation, if the power supplied from the power supply apparatus is higher than a power used in the radiation imaging apparatus, the power controller is configured to charge the internal power supply.

11. The radiation imaging system according to claim 7, wherein
the radiation imaging apparatus further comprises a load circuit unit, and
in the period of the first readout operation and the period of the second readout operation, if the power supplied from the power supply apparatus is higher than the power used in the radiation imaging apparatus, the power controller is configured to supply the power to the load circuit unit.

12. The radiation imaging system according to claim 11, wherein
in the period of the first readout operation and the period of the second readout operation, if the power supplied from the power supply apparatus is higher than the power used in the radiation imaging apparatus and a charge capacity of the internal power supply is a maximum value, the power controller is configured to supply the power to the load circuit unit.

13. The radiation imaging system according to claim 11, wherein
the load circuit unit converts the supplied power into heat.

14. The radiation imaging system according to claim 11, wherein
in the period of the first readout operation and the period of the second readout operation, if the power supplied from the power supply apparatus is higher than the power used in the radiation imaging apparatus, the power controller is configured to charge the internal power supply or supply the power to the load circuit unit such that the power used in the radiation imaging apparatus and the power supplied from the power supply apparatus become equal to each other.

15. The radiation imaging system according to claim 14, wherein
the radiation imaging apparatus comprises a plurality of the internal power supplies, and
the plurality of the internal power supplies include an internal power supply detachable from the radiation imaging apparatus and an internal power supply undetachable from the radiation imaging apparatus.

16. The radiation imaging system according to claim 1, wherein
the radiation imaging apparatus is configured to
perform a first imaging operation of accumulating electric charges in the sensor unit during radiation irradiation and then performing the first readout operation, and a second imaging operation of accumulating electric charges in the sensor unit without radiation irradiation and then performing the second readout operation, and
continuously perform the first imaging operation and the second imaging operation in the order of the first imaging operation and then the second imaging operation or in the order of the second imaging operation and then the first imaging operation.

17. The radiation imaging system according to claim 16, wherein
in the subsequent imaging operation of the first imaging operation and the second imaging operation, a reset operation of resetting the sensor unit is performed before accumulating electric charges in the sensor unit.

18. The radiation imaging system according to claim 1, wherein
the radiation imaging apparatus is configured to
perform a first operation of continuously performing first imaging operations, in each of which electric charges are accumulated in the sensor unit during radiation irradiation and then the first readout operation is performed, and a second operation of performing, at least once, a second imaging operation of accumulating electric charges in the sensor unit without radiation irradiation and then performing the second readout operation, and
continuously perform the second operation and then the first operation in this order.

19. The radiation imaging system according to claim 16, wherein
a time of accumulating electric charges in the sensor unit in the first imaging operation and a time of accumulating electric charges in the sensor unit in the second imaging operation are different from each other.

20. The radiation imaging system according to claim 1, wherein
the controller is arranged in the radiation imaging apparatus.

21. A control apparatus configured to control a radiation imaging apparatus, which comprises a sensor unit for obtaining a radiation image and is capable of non-contact power reception, and a power supply apparatus capable of non-contact power supply to the radiation imaging apparatus, wherein
the radiation imaging apparatus is configured to perform a first readout operation of reading out, from the sensor unit, a signal accumulated in a period during which radiation irradiation is performed, and a second readout operation of reading out, from the sensor unit, a signal accumulated in a period during which no radiation irradiation is performed, and
the control apparatus is configured to control the radiation imaging apparatus and the power supply apparatus such that the power supplied from the power supply apparatus to the radiation imaging apparatus in a period of a succeeding operation, which is the succeedingly performed readout operation of the first readout operation and the second readout operation, temporally changes in accordance with a temporal change in the power supplied from the power supply apparatus to the radiation imaging apparatus in a period of a preceding operation, which is the precedingly performed readout operation of the first readout operation and the second readout operation.
